(19) United States
(12) Patent Application Publication
(10) Pub. No.: US 2026/0280596 A1
(43) Pub. Date: Sep. 17, 2026

(54) EMERGENCY MEDICAL DATA AUDIO BROADCAST DEVICE

(71) Applicant: Linda Clark, Middletown, DE (US)

(72) Inventor: Linda Clark, Middletown, DE (US)

(21) Appl. No.: 19/081,479

(22) Filed: Mar. 17, 2025

(51) Int. Cl. H04B 1/3822; G08B 25/10; H04R 1/02

(52) U.S. Cl. CPC H04B 1/3822; G08B 25/10; H04R 1/025

(57) ABSTRACT

An emergency medical data audio broadcast device for broadcasting emergency medical data to assist first responders in the event of a vehicular accident includes a housing having an interior space. A processor is positioned in the interior space. A is speaker coupled to the housing and operationally coupled to the processor. A memory is positioned in the interior space for storing a recorded audio of medical data. The memory is operationally coupled to the processor. An actuator is positioned on the housing and is operationally coupled to the processor to initiate broadcast of the recorded audio of medical data upon manipulation of the actuator.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to audio broadcasting devices and more particularly pertains to a new audio broadcasting device for automatically broadcasting emergency medical data to assist first responders in the event of a vehicular accident.

### (2) Description of Related Art including information disclosed under 37 CFR 1.97 and 1.98

The prior art relates to audio broadcasting devices but lacks provision for a housing dedicated to coupling to a child car seat and providing broadcast of medical data upon pressing a button directly into the housing by always pressing in a direction into the child car seat structure.

### (h) BRIEF SUMMARY OF THE INVENTION

An embodiment of the disclosure meets the needs presented above by generally comprising a housing having an interior space. A processor is positioned in the interior space. A is speaker coupled to the housing and operationally coupled to the processor. A memory is positioned in the interior space for storing a recorded audio of medical data.

The memory is operationally coupled to the processor. An actuator is positioned on the housing and is operationally coupled to the processor to initiate broadcast of the recorded audio of medical data upon manipulation of the actuator.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front side perspective view of an emergency medical data audio broadcast device according to an embodiment of the disclosure.

FIG. 2 is a front view of an embodiment of the disclosure.

FIG. 3 is a rear view of an embodiment of the disclosure.

FIG. 4 is a top view of an embodiment of the disclosure.

FIG. 5 is a top front side perspective view of an embodiment of the disclosure installed.

FIG. 6 is a side view of an embodiment of the disclosure in use.

FIG. 7 is a schematic view of an embodiment of the disclosure.

### (j) DETAILED DESCRIPTION OF THE INVENTION

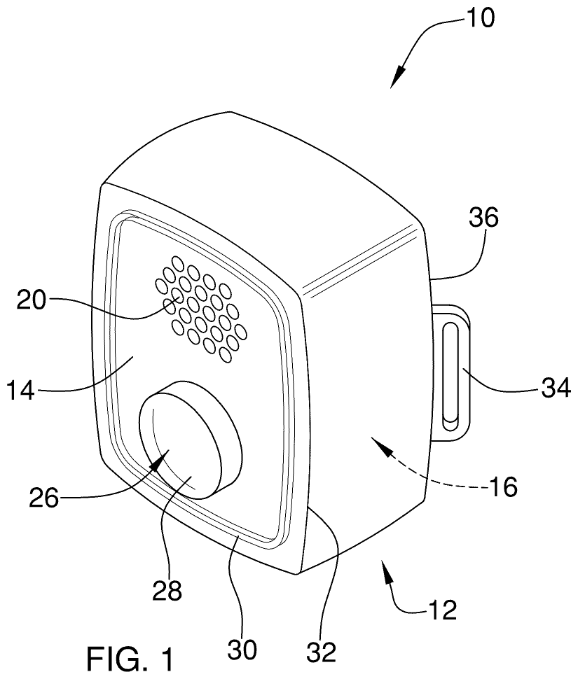

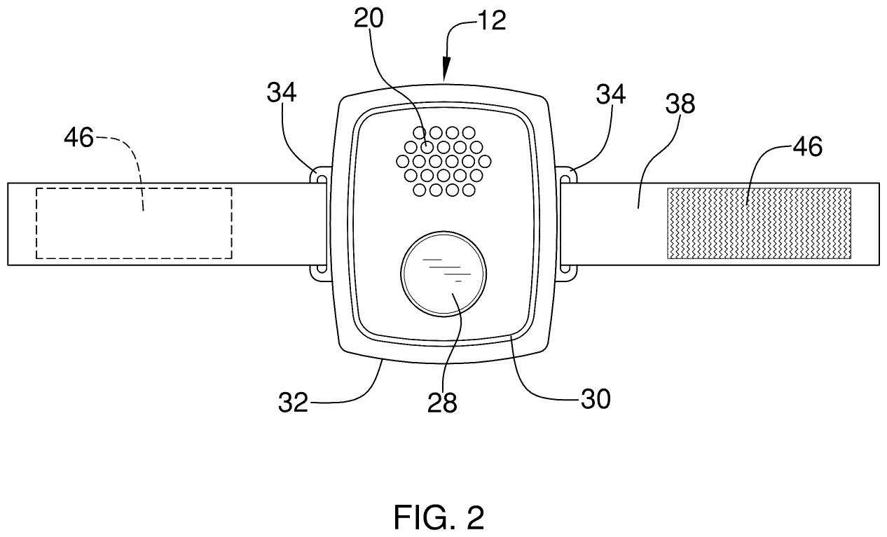

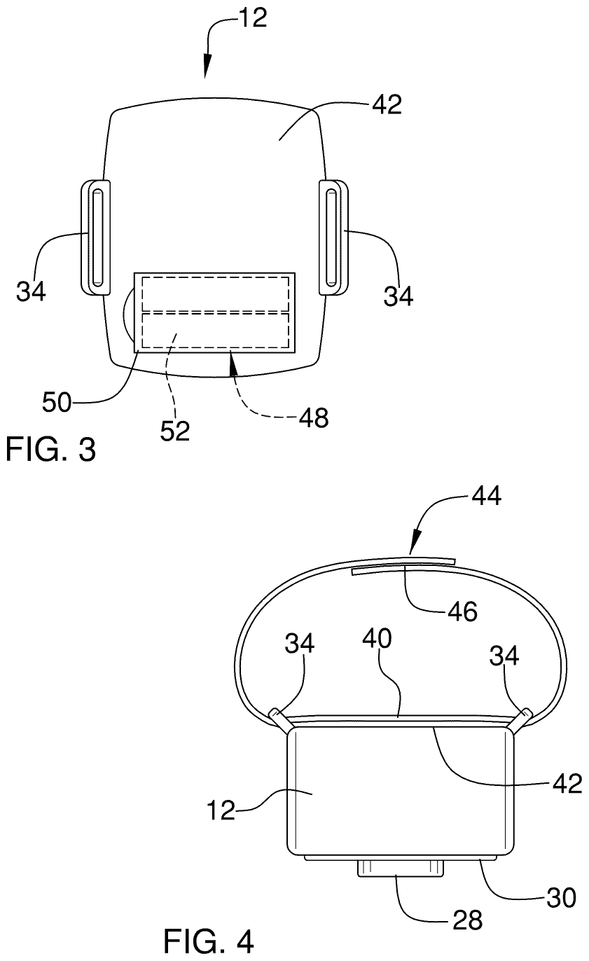

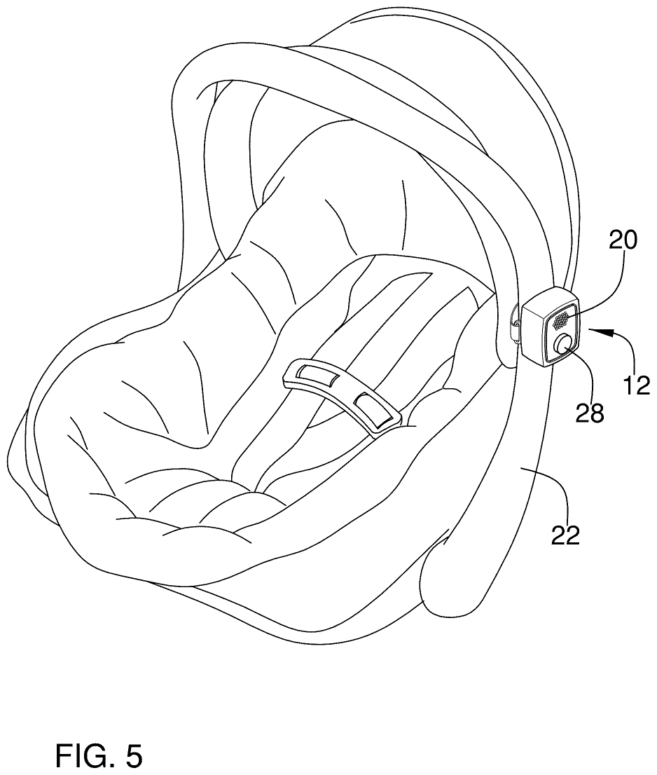

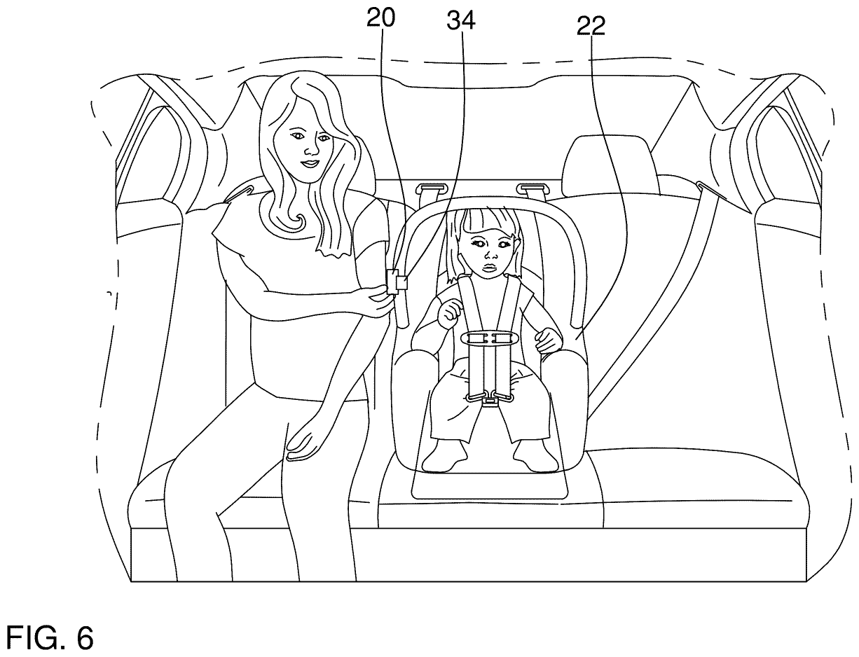

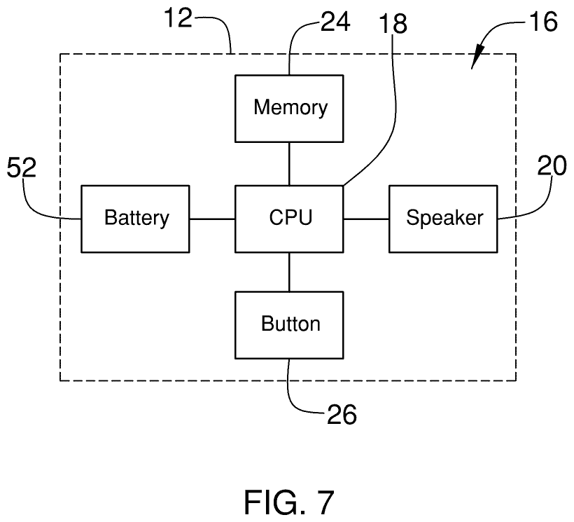

With reference now to the drawings, and in particular to FIGS. 1 through 7 thereof, a new audio broadcasting device embodying the principles and concepts of an embodiment of the disclosure and generally designated by the reference numeral 10 will be described.

As best illustrated in FIGS. 1 through 7, the emergency medical data audio broadcast device 10 generally comprises a housing 12 having a front face 14 and an interior space 16. A processor 18 is positioned in the interior space 16. A speaker 20 is coupled to the housing 12. The speaker 20 is operationally coupled to the processor 18 and is positioned on the front face 14 of the housing 12. Thus, the speaker 20 is configured to face away from a child car seat 22 to which the housing may be attached as described further below.

A memory 24 is positioned in the interior space 16 for storing a recorded audio of medical data. The medical data may be any medical condition, allergies, blood type, or other information which might be needed by first responders in the event of an accident. The memory 24 is operationally coupled to the processor 18. An actuator 26 is positioned on the housing 12 and is operationally coupled to the processor 18. Activation or manipulation of the processor 18 initiates broadcast of the recorded audio of medical data. The actuator 26 is more specifically a button 28 activated by being pressed into the housing 12. The button 28 is positioned on the front face 14 of the housing 12 such that pressing the button 28 into the housing 12 is facilitated by resistance of the housing 12 due to being coupled to the child car seat 22 or other structure as described below. A ridge 30 is coupled to and extends forwardly from the front face 14 of the housing 12.

The ridge 30 encircles the actuator 26 and the speaker 20. A length of the button 28 extending from the front face 14 is greater than a length the ridge 30 extends from the front face 14 such that the button 28 protrudes and is easy to locate by touch. The ridge 30 is inset from a peripheral edge 32 of the front face 14 of the housing 12.

Each of a pair of connections loops 34 is coupled to and extends from the housing 12. Each connection loop 34 is positioned along a respective opposing edge 36 of the housing 12. Each of the connection loops 34 extends outwardly and rearwardly from the housing 12 such that the pair of connection loops 34 are flared extending from the housing 12. A connection strap 38 extends through each connection loop 34 of the pair of connection loops 34. The connection strap 38 extends through both of the connection loops 34 such that a medial section 40 the connection strap 38 is parallel to and abuts a rear face 42 of the housing 12. A fastener 44 is positioned on the connection strap 38 for securing the housing 12 to the child car seat 22, or similar structure in a desired location. The fastener 44 may be complementary portions of hook and loop fastener 46, a buckle, or the like.

A battery compartment 48 is positioned in the housing 12. The battery compartment 48 is positioned on and accessed through the rear face 42 of the housing 12. Thus, a cover 50 of the battery compartment 48 is configured to be positioned abutting the child car seat 22, or other structure, when the housing 12 is secured to the child car seat 22. This facilitates retention of the cover 50 and prevents inadvertent opening of the battery compartment 48. A battery 52 is positioned in the battery compartment 48. The battery 52 is electrically coupled to the processor 18. The battery 52 may be rechargeable either by removal from the battery compartment 48 or through connection of a power cord to a charging port on the housing 12.

In use, the housing 12 is attached to the child car seat 22 or whatever structure for use at whatever desired location. In the event of an accident, particularly one involving a vehicle where occupants of the vehicle may not be able to speak, first responders, or others arriving at the accident site may press the button 28 at which time the audio recording of medical data will be broadcast. Thus, the first responders may be made aware of information which could assist in decision making as to how to respond and assist occupants of the vehicle.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. An emergency medical data audio broadcast device comprising:
a housing having an interior space;
a processor positioned in said interior space;
a speaker coupled to said housing, said speaker being operationally coupled to said processor;
a memory positioned in said interior space for storing a recorded audio of medical data, said memory being operationally coupled to said processor; and
an actuator positioned on said housing, said actuator being operationally coupled to said processor wherein said processor initiates broadcast of said recorded audio of medical data upon manipulation of said actuator.

2. The emergency medical data audio broadcast device of claim 1, further comprising:
a connection loop coupled to and extending from said housing;
a connection strap extending through said connection loop; and
a fastener positioned on said connection strap wherein said connection strap is configured for securing said housing to a child car seat.

3. The emergency medical data audio broadcast device of claim 2, wherein said fastener is complementary portions of hook and loop fastener.

4. The emergency medical data audio broadcast device of claim 2, wherein said connection loop is one of a pair of connection loops, each connection loop being positioned along a respective opposing edge of said housing, said connection strap extending through both of said connection loops such that said connection strap is parallel to and abuts a rear face of said housing.

5. The emergency medical data audio broadcast device of claim 4, further comprising each of said connection loops extending outwardly and rearwardly from said housing such that said pair of connection loops are flared extending from said housing.

6. The emergency medical data audio broadcast device of claim 1, further comprising said actuator being a button activated by being pressed into said housing.

7. The emergency medical data audio broadcast device of claim 6, further comprising said button being positioned on a front face of said housing.

8. The emergency medical data audio broadcast device 1, further comprising said speaker being positioned on a front face of said housing wherein said speaker is configured to face away from the child car seat.

9. The emergency medical data audio broadcast device of claim 7, further comprising:
said speaker being positioned on a front face of said housing wherein said speaker is configured to face away from the child car seat; and
a ridge coupled to and extending forwardly from said front face of said housing, said ridge encircling said actuator and said speaker.

10. The emergency medical data audio broadcast device of claim 9, further comprising a length of said button extending from said front face being greater than a length said ridge extends from said front face.

11. The emergency medical data audio broadcast device of claim 9, further comprising said ridge being inset from a peripheral edge of said front face of said housing.

12. The emergency medical data audio broadcast device of claim 1, further comprising:
a battery compartment positioned in said housing; and
a battery positioned in said battery compartment, said battery being electrically coupled to said processor.

13. The emergency medical data audio broadcast device of claim 12, further comprising said battery compartment being positioned on and accessed through a rear face of said housing wherein a cover of said battery compartment is configured to be positioned abutting the child car seat when the housing is secured to the child car seat.

14. An emergency medical data audio broadcast device comprising:
a housing having a front face and an interior space;
a processor positioned in said interior space;
a speaker coupled to said housing, said speaker being operationally coupled to said processor, said speaker being positioned on said front face of said housing wherein said speaker is configured to face away from the child car seat, ;
a memory positioned in said interior space for storing a recorded audio of medical data, said memory being operationally coupled to said processor;
an actuator positioned on said housing, said actuator being operationally coupled to said processor wherein said processor initiates broadcast of said recorded audio of medical data upon manipulation of said actuator, said actuator being a button activated by being pressed into said housing, said button being positioned on said front face of said housing;
a ridge coupled to and extending forwardly from said front face of said housing, said ridge encircling said actuator and said speaker, a length of said button extending from said front face being greater than a length said ridge extends from said front face, said ridge being inset from a peripheral edge of said front face of said housing;
a pair of connections loops coupled to and extending from said housing, each connection loop being positioned along a respective opposing edge of said housing, each of said connection loops extending outwardly and rearwardly from said housing such that said pair of connection loops are flared extending from said housing;
a connection strap extending through each connection loop of said pair of connection loops, said connection strap extending through both of said connection loops such that said connection strap is parallel to and abuts a rear face of said housing;
a fastener positioned on said connection strap wherein said connection strap is configured for securing said housing to a child car seat, said fastener being complementary portions of hook and loop fastener;
a battery compartment positioned in said housing, said battery compartment being positioned on and accessed through said rear face of said housing wherein a cover of said battery compartment is configured to be positioned abutting the child car seat when the housing is secured to the child car seat; and
a battery positioned in said battery compartment, said battery being electrically coupled to said processor.
